(19) United States
(12) Patent Application Publication
(10) Pub. No.: US 2003/0003576 A1
(43) Pub. Date: Jan. 2, 2003

(54) Peripheral blood fibrocytes differentiation pathway and migration to wound sites

(76) Inventors: Riichiro Abe, Sapporo (JP); Richard Bucala, Cos Cob, CT (US); Seamas Donnelly, Dublin (IE); Christine N. Metz, Great Neck, NY (US)

Correspondence Address: Supervisor, Patent Prosecution Services, PIPER RUDNICK LLP, 1200 Nineteenth Street, N.W., Washington, DC, 20036-2412, US

(21) Appl. No.: 10/160,048

(22) Filed: Jun. 4, 2002

Related U.S. Application Data
(60) Provisional application No. 60/294,988, filed on Jun. 4, 2001.

(51) Int. Cl. C12N 5/08

(52) U.S. Cl. 435/372

(57) ABSTRACT

Disclosed are the identification of a differentiation pathway of cultured fibrocytes, characterization of the signals for fibrocyte migration to wound sites in vivo, and the potential role of fibrocytes in wound contracture. The invention relates to a method for producing fibrocytes comprising contacting a population of human peripheral blood mononuclear cells (PBMC) comprising predominantly CD14⁺ cells with autologous T cells or a form of TGFβ, preferably TGFβ₁, thereby inducing differentiation of fibrocytes from precursors in the PBMC population. These fibrocytes are useful for treating a wound in a mammalian subject by administering fibrocytes to the subject, preferably in combination with TGFβ₁. Also disclosed are methods for attracting or targeting fibrocytes to a wound by administering SLC or another agonist of the CCR7 chemokine receptor, at or near the site of the wound, and methods of decreasing undesired wound fibrosis by inhibiting fibrocyte activity.

This application claims priority from U.S. Provisional Application Serial No. 60/294,988 filed Jun. 4, 2001. The entirety of that provisional application is incorporated herein by reference.

### BACKGROUND OF THE INVENTION

1. Field of the Invention

The present invention relates to methods and compositions for the production, use and inhibition of fibrocytes, including: producing fibrocytes ex vivo, particularly using T cells or TGFβ; targeting fibrocytes to a wound in vivo using a ligand of the CCR7 chemokine receptor, particularly secondary lymphoid chemokine (SLC); and decreasing fibrocyte effects, for instance in undesired wound fibrosis, by interfering with fibrocyte activity, particularly by using an inhibitor of SLC activity.

2. Background of the Technology

Fibroblasts, depending on their tissue source and stimuli for activation, are a heterogenous population of cell types exhibiting distinct functions. Fibroblasts found in the wound are considered important for the healing process. The concept that wound fibroblasts can originate from peripheral blood cells goes back almost 100

years (reviewed in 1). Since then, numerous studies have reported the differentiation of peripheral mononuclear cells into fibroblast-like cells.

In 1994, a distinct population of blood-borne fibroblast-like cells that rapidly enter sites of tissue injury was described (2). Termed ‘fibrocytes’, these cells comprise 0.1-0.5% of non-erythrocytic cells in peripheral blood and display an adherent, spindle-shaped morphology when cultured in vitro. Cultured fibrocytes express the fibroblast products collagen I, collagen III, and fibronectin, as well as the leukocyte common antigen (CD45RO), the pan-myeloid antigen (CD13), and the hemopoietic stem cell antigen (CD34). In addition, fibrocytes express MHC class II and co-stimulatory molecules (CD80 and CD86) and have the capacity to present antigen in vitro and in vivo (3,4). By their morphology, growth properties, and cell surface markers, fibrocytes appear to be distinct from monocytes/macrophages, dendritic cells, and other known antigen-presenting cell types. Cultured fibrocytes do not express typical monocyte/macrophage-specific or B cell markers (such as CD14, CD16, or CD19). nor do they express typical surface proteins of dendritic cells or their precursors (such as CD1a, CD10, CD25, and CD38). In addition, fibrocytes isolated from peripheral blood and cultured ex vivo secrete a unique profile of cytokines, growth factors and chemokines (5).

Based on their presence in wounds and their secretion of pro-inflammatory cytokines, chemokines, and extracellular matrix proteins, fibrocytes have been postulated to play a role in wound healing and connective tissue formation. Although initial studies performed in sex-mismatched bone marrow chimeric mice suggested that fibrocytes arose from a relatively radio-resistant progenitor population (2), the precise origin of these cells and the wound trafficking signals relevant to their directed migration remain unknown.

U.S. Pat. No. 5,804,446 to Cerami et al. discloses “blood-borne mesenchymal cells” including fibrocytes, and methods for producing and using such cells. U.S. Pat. No. 6,153,441 to Appelbaum et al., discloses methods for screening for discovering agonists and antagonists of the interaction between a secreted human protein, chemokine CKβ-9 (also known as secondary lymphoid chemokine (SLC), Exodus-2, 6Ckine and TCA-4) and its cellular receptor, human CCR7 (also known as EBI1 and BLR2).

### SUMMARY OF THE INVENTION

Fibrocytes are a distinct population of blood-borne cells that display a unique cell surface phenotype (collagen I⁺/CD11b⁺/CD13⁺/CD34⁺/CD45RO⁺/MHC Class II⁺/CD86+) and exhibit potent immunostimulatory activities. The present invention is based in part upon identification of a differentiation pathway of cultured fibrocytes, characterization of the signals for fibrocyte migration to wound sites in vivo, and revelation of the potential role of fibrocytes in wound contracture.

As reported herein, ex vivo cultured fibrocytes can differentiate from a CD14⁺-enriched mononuclear cell population, and this process requires contact with T cells. Further, TGFβ (1-10 ng/ml), an important fibrogenic and growth-regulating cytokine involved in wound healing, increases the differentiation and functional activity of cultured fibrocytes. These findings provide a mechanism for a differentiation pathway of cultured fibrocytes and identify TGFβ, which has been previously implicated in signaling and accessory functions for immune cell activation, as a natural fibrocyte differentiation factor.

Accordingly, one aspect of the present invention relates to a method for producing fibrocytes comprising contacting a population of human peripheral blood mononuclear cells (PBMC) comprising predominantly CD14⁺ cells with autologous T cells, preferably for a period of 7-10 days, thereby inducing differentiation of fibrocytes from precursors in the PBMC population. In this method, the population of predominantly CD14⁺ cells may be provided, for instance, by cultivation of an adherent PBMC population on a solid substrate. The CD14⁺ cells may be further purified before or after contact with the T cells, for instance, by removal of T or B cell populations, using antibodies to cell surface antigens.

In an alternative method of the invention, fibrocytes are produced by inducing differentiation of fibrocytes from a PBMC population, preferably a population of predominantly CD14⁺ cells and optionally in contact with T cells, by contacting the population with a form of TGFβ, preferably TGFβ₁. Preferably, the PBMC population is cultured with 1-10 ng/ml TGFβ₁ for several days, for instance, a week. Optionally, CD14⁺ cells are purified from the PBMC population after culturing with TGFβ₁.

A wide variety of uses of the fibrocytes, and factors produced by these cells, are encompassed by the invention described herein, particularly to improve wound healing, including, but not limited to, cutaneous wounds, corneal wounds, wounds of epithelial-lined organs, resulting from physical abrasions, cuts, burns, chronic ulcers, inflammatory conditions and the like, as well as from any surgical procedure. In one embodiment of the invention, the fibrocytes produced by the method of the invention are useful, for instance, in a method of treating a wound in a mammalian subject, preferably a human subject, comprising administering fibrocytes produced by the invention to the subject. Preferably, the fibrocytes are administered to such a subject in combination with TGFβ₁, where the fibrocytes and TGFβ₁ are administered in a composition comprising the cells and the TGFβ₁ or in separate compositions or both. Fibrocytes prepared by the invention method, and optionally TGFβ₁, are administered systemically, for instance parenterally, such as by intravenous injection, or locally, such as topically on an exposed wound, or subdermally or intraperitoneally.

As further reported herein, in studying why fibrocytes home to sites of tissue injury, it was discovered that secondary lymphoid chemokine (SLC), a ligand of the CCR7 chemokine receptor, acts as a potent stimulus for fibrocyte chemotaxis in vitro and for the homing of injected fibrocytes to sites of cutaneous tissue injury in vivo.

Accordingly, the present invention relates to a method for purifying or enriching for fibrocytes comprising exposing a fibrocyte-containing mixed cell population to a gradient of SLC such that fibrocytes separate themselves from other cell types in the mixed cell population.

In another aspect the present invention relates to a method for attracting or targeting fibrocytes to a wound in a mammalian subject, preferably a human subject, by administering SLC or another agonist of the CCR7 chemokine receptor to the subject, at or near the site of the wound. The SLC or other agonist of the CCR7 chemokine receptor is administered, for instance, locally, such as topically on an exposed wound or intradermally, subdermally or intraperitoneally at or near the site of an unexposed wound. Preferably, the SLC is administered in a unit dosage of from about 100 ng to about 1 mg/dose, preferably about 1 μg to about 100 μg, at least once a day, more preferably several times per day, until the desired wound healing is obtained. Thus, SLC may be administered for a period of at least about three days to about one week, or for several weeks or more, depending on how quickly the desired healing is obtained.

Agonists of the CCR7 chemokine receptor other than SLC may be isolated using the known CCR7 chemokine receptor and methods known in the art for isolating receptor agonists, for instance, by systematic mutational analysis of SLC or by known approaches for identifying small molecule mimetics of a polypeptide such as the chemokine SLC, using methods known in the art.

The method of the invention for attracting or targeting fibrocytes to a wound optionally may be combined with the above method of treating a wound using fibrocytes produced by an invention method, by administering fibrocytes produced by the invention to a subject having a wound, optionally in combination with TGFβ₁, either before, after or preferably concurrently with administration of SLC at or near a wound site.

In another aspect the present invention relates to methods of decreasing undesired effects of fibrocytes, such as undesired wound fibrosis by inhibiting fibrocyte activity. In one embodiment of this method of inhibiting undesired wound fibrosis, an inhibitor of fibrocyte activity is administered to a mammalian subject, preferably a human subject, having a wound that exhibits or is expected to exhibit undesired fibrosis. The inhibitor of fibrocyte activity is administered systemically, for instance parenterally, such as by intravenous injection, preferably locally, at or near the site of the wound, such as intraperitoneally, or topically on an exposed wound, or intra- or subdermally. The inhibitor of fibrocyte activity used in this aspect of the invention is selected from the group consisting of agents that interfere with stimulation of fibrocyte differentiation by T cells, agents that interfere with stimulation of fibrocyte differentiation by TGFβ₁, and agents that interfere with attraction of fibrocytes by SLC. Optionally, a method of decreasing undesired effects of fibrocytes of this invention employs a combination of one or more agents that interfere with stimulation of fibrocyte differentiation by T cells or by TGFβ₁, and/or agents that interfere with attraction of fibrocytes by SLC.

Agents that interfere with stimulation of fibrocyte differentiation by T cells may be identified, for instance, using a cell culture assay for T cell stimulation of fibrocyte differentiation based on methods disclosed herein, and include without limitation, antibodies to T cells that interfere with stimulation of fibrocyte differentiation. Agents that interfere with stimulation of fibrocyte differentiation by TGFβ₁ include, by way of non-limiting example, antibodies that inhibit stimulation of fibrocyte differentiation by preventing TGFβ₁ from binding to a fibrocyte receptor for TGFβ₁ including, by way of example, but not limitation, antibodies that bind either to TGFβ₁, or to the fibrocyte receptor for TGFβ₁.

Agents that interfere with attraction of fibrocytes by SLC include agents that interfere with production of SLC and agents that interfere with the activity of SLC, including, for instance, antibodies that inhibit attraction of fibrocytes by preventing SLC from binding to a fibrocyte (CCR7 chemokine) receptor for SLC, such as antibodies that bind either to SLC or to the fibrocyte receptor for SLC. Agents that interfere with the activity of SLC that may be used in this invention method also include a soluble SLC receptor or fragment thereof that binds SLC, and an antagonist or competitive inhibitor of SLC that competes with SLC for binding to the fibrocyte SLC receptor but does not activate that receptor or activates that receptor to a lesser extent than SLC.

### DESCRIPTION OF THE FIGURES

FIG. 1 shows that fibrocytes differentiate in vitro from a blood-derived CD14⁺ population and require direct T cell interaction. (A) Schematic representation of the experimental design. Adherent cells from a human PBMC fraction isolated from whole blood were collected after an overnight incubation (designated “Total”). A CD14⁺-enriched population was isolated from the “Total” fraction by depletion of T and B cells (designated “CD14⁺”) and a CD14⁻-enriched population was isolated from the “Total” fraction by depletion of CD14⁺ monocytes (designated “CD14⁻”). (B) Using the Transwell™ culture system (0.4 μm), total adherent PBMCs (“Total”), CD14⁺-enriched cells or total PBMCs depleted of T and B cells (“CD14⁺”), and total PBMCs depleted of CD14⁺ cells (“CD14⁻”) were cultured in the upper and lower chambers, as indicated, for 7 days. Cells in the lower chambers were lifted and analyzed for fibrocyte phenotype using CD11b⁺/collagen I⁺ staining by flow cytometry and data are represented as the % fibrocytes. (C) CD14⁺-enriched cells (PBMCs depleted of both T and B cells) were incubated with various ratios of autologous T cells and the resulting “crude” fibrocyte cultures were analyzed for fibrocyte markers after 7 days in culture by flow cytometry. Data show the % fibrocytes based on CD11b⁺/collagen I⁺ staining.

FIG. 2 shows that TGFβ₁ promotes the differentiation of fibrocytes. Four days following their isolation from human blood, “crude” fibrocyte cultures were treated with various concentrations of TGFβ₁. Then 7 days later, cultures were examined for spindle-shaped morphology. Representative cultures photographed at 200× are shown: (A) no addition, (B) TGFβ₁, 1 ng/ml, (C) TGFβ₁, 10 ng/ml. (D) TGFβ₁, treated (0-10 ng/ml) “crude” fibrocyte cultures were lifted, stained for collagen I, and analyzed by flow cytometry. Isotype control staining of cells is shown as shaded histogram. The Y-axis represents relative cell number and X-axis represents mean fluorescence intensity (collagen I staining).

FIG. 3 shows that fibrocytes migrate to wound sites in vivo. Cultured, “enriched” mouse fibrocyte preparation (>96% pure) were labeled with the fluorescent dye, PKH-26. Labeled cells (5×10⁵) were injected into the tail vein of BALB/c mice. Immediately following injection of the fibrocytes, a single full-thickness round skin wound was made in the dorsal subscapular area of each mouse. After 4 days, mice were sacrificed and wound sites were removed. The migration of labeled fibrocytes was assessed by (A) fluorescent microscopic examination of thin frozen sections of the wound (left panel); right panel shows H&E staining of a similar wound site; or (B) by quantitative cytofluorometric analysis of the number of fluorescent fibrocytes found in the biopsies of wounded skin vs. non-wounded skin (with and without i.v. injection of fluorescent fibrocytes) following proteolytic dissociation of 250 μg biopsy sites.

FIG. 4 shows that human fibrocyte preparations express CCR3, CCR5, CCR7, and CXCR4 mRNA and protein. (A) RT-PCR was used to determine mRNA expression for various chemokine receptors by cultured, “enriched” human fibrocyte preparations. (B) Cultured, “enriched” human fibrocytes were stained for surface expression with anti-CCR3, CCR5, CCR7, or CXCR4 antibodies, and then analyzed by flow cytometry. Shaded region represents isotype control staining. (C) Cultured, “enriched” mouse fibrocyte preparations were stained for surface expression with anti-CCR7 and CXCR4 antibodies, and then analyzed by flow cytometry.

FIG. 5 shows that fibrocytes migrate in response to SLC in vitro and in vivo. (A) SLC and SDF chemokines, or buffer alone diluted in DMEM 1% BSA were added to individual wells of a 24-well plate at the indicated final concentrations. Immediately thereafter, Costar Transwell™ devices were inserted, and cultured, “enriched” mouse fibrocyte preparations (400 μl in DMEM 1% BSA at 10⁶ cells/ml) were layered on top of the membrane (8 μm pore size). Cells were allowed to migrate through the membrane for 3 h at 37° C. Transmigrated cells were collected and counted by flow cytometry. The number of cells migrating to the lower chamber is presented as a % of the total number of fibrocytes added to the upper well. *, p<0.05 as determined by Student's t test comparing experimental (at indicated concentrations) vs. media alone (not shown; <2%). (B) For checkboard-type analysis, SLC (100 ng/ml) was added to the upper and/or lower wells as indicated and in vitro chemotaxis of fibrocytes was performed as described above. (C) Immediately following tail vein injection of PKH-26-labeled cultured, “enriched” mouse fibrocytes (5×10⁵), mice-received either an i.d. injection of SLC or of SDF (0.1 or 1 μg, in 50 μl) or PBS vehicle. Injected sites (250 μg) were surgically removed 4 hrs later and proteolytically digested to obtain a single cell suspension. The number of labeled fibrocytes per injection site was quantified by flow cytometry and expressed as a % of the total number of fluorescent fibrocytes injected into the tail vein. *, p<0.05 as determined by Student's t test comparing SLC injected at 1 pg vs. vehicle injection.

FIG. 6 shows that fibrocytes express α-smooth-muscle actin (αSMA) and contract collagen gels in vitro. Expression of αSMA mRNA by adherent human PBMCs, cultured, “enriched” fibrocytes (FCs), and human intestinal smooth muscle (HISM) cells, as analyzed by RT-PCR. Stds=DNA bp ladder. (B) Expression of intracellular αSMA expression by unstimulated and TGFβ₁(10 ng/ml) treated cultured, “enriched” human fibrocyte preparations, as determined by flow cytometry. (C) Collagen gel contraction assay. PBMCs (Δ), cultured, “enriched” fibrocyte preparations [untreated (♦) and TGFβ₁-treated (▪)], or dermal fibroblasts (∘) were resuspended in a collagen type I solution at 10⁵ cells/ml. The contraction assay (n=3) was performed as described in Materials and Methods. The data represent the % gel contraction (from beginning of experiment) ±SE (some error bars are smaller than symbol). * p<0.05, as determined by Student's t test, comparing experimental to PBMCs (Δ) at each time point. Inset shows representative contracted gels after incubation with PBMCs vs. cultured fibrocytes (untreated and TGFβ₁-treated).

FIG. 7 illustrates a proposed differentiation pathway of fibrocytes from a circulating precursor population.

### DETAILED DESCRIPTION

Previous studies have shown that fibrocytes, a distinct mesenchymal cell type that arises in ex vivo cultures of peripheral blood, exhibit both monocyte and fibroblast-like characteristics (reviewed in 21). Fibrocytes initially were identified by their rapid and specific recruitment from the blood to subcutaneously implanted wound chambers in mice (2). Human fibrocytes then were shown to emerge from cultures of the PBMC fraction of whole blood after a week or two (2). Cultured fibrocytes have been shown to mediate fibrosis (5), antigen-presentation and immunity (3,4), and angiogenesis (CNM, unpublished data). In the present study, the differentiation pathway of peripheral blood fibrocytes was examined and the role of fibrocytes in wound repair was explored.

Fibrocytes differentiated from an adherent population of CD14⁺-enriched peripheral blood cells when cultured in DMEM and FBS (with no additional growth factors). See EXPERIMENTAL, below. This differentiation process was significantly enhanced by T cell interaction.

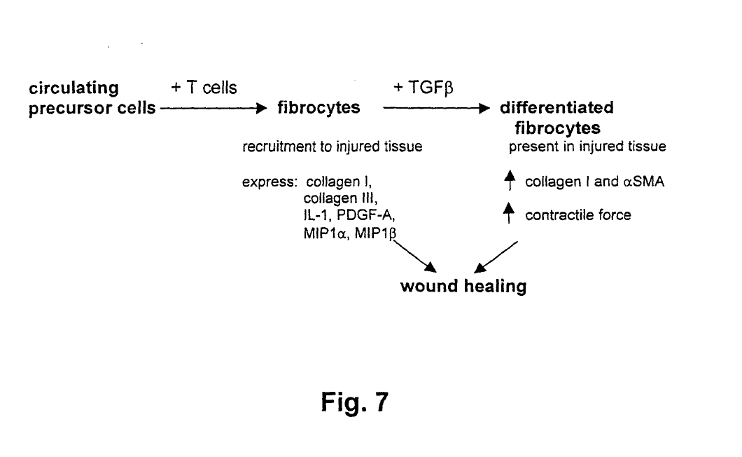

Interestingly, the addition of TGFβ₁, a multifunctional cytokine that plays a central role in tissue repair and fibrosis, to “crude” fibrocyte-evolving cultures facilitated fibrocyte differentiation. The role of exogenous TGFβ on fibroblast proliferation and collagen production is well-documented (reviewed in 2). TGFβ significantly up-regulates collagen expression by dermal fibroblasts in vitro (29), by myofibroblasts (30), as well as by proliferative scar xenografts in vivo (31). Many laboratories have confirmed that TGFβ plays a role in the natural wound healing process and that TGFβ is expressed in rodent wound chambers during the early-mid phases (days 4-7) of wound healing (32). Furthermore, in vivo gene transfer with TGFβ₁, cDNA into the skin of rats significantly enhanced the rate of wound repair (33). Consistent with these prior observations, we postulate that circulating fibrocyte precursor cells interact with activated T cells which permits their early differentiation (toward the fibrocyte phenotype), and then they migrate to the wound site (FIG. 7). Within the wound site, these “early” differentiated fibrocytes might further interact with recruited T cells, and fully differentiate and mature following exposure to TGFβ. These fully differentiated, mature fibrocytes express increased levels of αSMA and produce collagen and other extracellular matrix proteins that promote wound healing and contracture.

Fibroblasts have been shown to exhibit increased collagen expression and other matrix components in certain fibrotic disease states (reviewed by 34). Investigators have previously implicated TGFβ overexpression in fibrosis of the skin (35) and lungs (35,36). In addition, TGFβ overexpression has been associated with enhanced myofibroblast activity in animal models of pulmonary fibrosis (37). Our findings that TGFβ₁ enhanced proliferation, collagen production, and αSMA expression by cultured fibrocytes potentially implicates this circulating cell type in TGFβ-dependent fibrotic responses in vivo.

A role for fibrocytes in wound healing and connective scar tissue formation has been postulated based on their accumulation in wound sites (2). However, the molecular signals that mediate the trafficking of fibrocytes to the wound has not yet been investigated. We examined chemokine receptor expression (mRNA and protein) by cultured “enriched” fibrocyte preparations and revealed the presence of CCR3, CCR5, CCR7, and CXCR4 and the absence of CCR4, CCR6, and CXCR3. Further studies showed directed chemotaxis of cells from cultured, “enriched” fibrocyte preparations in response to the ligand of CCR7, SLC (also known as 6Ckine, Exodus-2, and TCA-4), in vitro and in vivo. SLC, a C-C chemokine family member, has been shown to be involved in the organization of lymphoid tissue during development by attracting T cells and mature dendritic cells (38). SLC expression has been observed in sites of inflammation (39). We observed SLC expression by the vascular endothelium within wound sites. Based on these observations, it would be interesting to examine the role of fibrocytes in wound responses using mutant mice lacking SLC expression (40-42).

The function of fibrocytes in wound healing has previously not been investigated. TGFβ has been shown to be the most important cytokine for the trans-differentiation of fibroblasts to contractile wound myofibroblasts which exhibit increased αSMA staining, elevated collagen secretion (reviewed in 19), and increased stress fibers (17) in response to TGFβ. Myofibroblasts are transiently found in ‘early-mid’ wound tissue and have been proposed to exert a critical contractile force that is required close wounds. Neither the origin of myofibroblasts nor any trafficking signals necessary for myofibroblast accumulation at sites of tissue injury are well understood. Myofibroblasts have been postulated to derive from either progenitor stem cells, resident tissue fibroblasts, or from tissue smooth muscle cells. However, a plausible alternative is that myofibroblasts differentiate from a circulating, rather than a resident, precursor cell type.

In this disclosure we show that blood-borne, ex vivo cultured, precursor fibrocyte cells have the capacity to differentiate into αSMA⁺, TGFβ₁-responsive fibrocyte cells that exhibited characteristics similar to wound-healing myofibroblasts. Differentiated fibrocytes and myofibroblasts share many common features: transient presence within the wound, production of numerous pro-inflammatory cytokines and growth factors, secretion of collagen and other extracellular matrix proteins, and enhanced collagen production in response to TGFβ₁. Furthermore, we observed that cultured fibrocytes, like myofibroblasts, express αSMA protein that is enhanced by TGFβ₁ treatment and, further, that cultured isolated fibrocytes exert a contractile force suited to reducing the amount of denuded surface area of wounded tissue. Thus, fibrocytes derived from a circulating precursor population play an important role during the resolution and repair phase of wound healing.

### EXPERIMENTAL

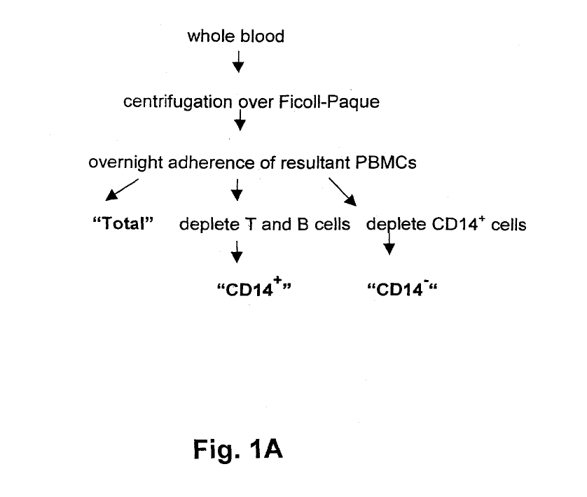

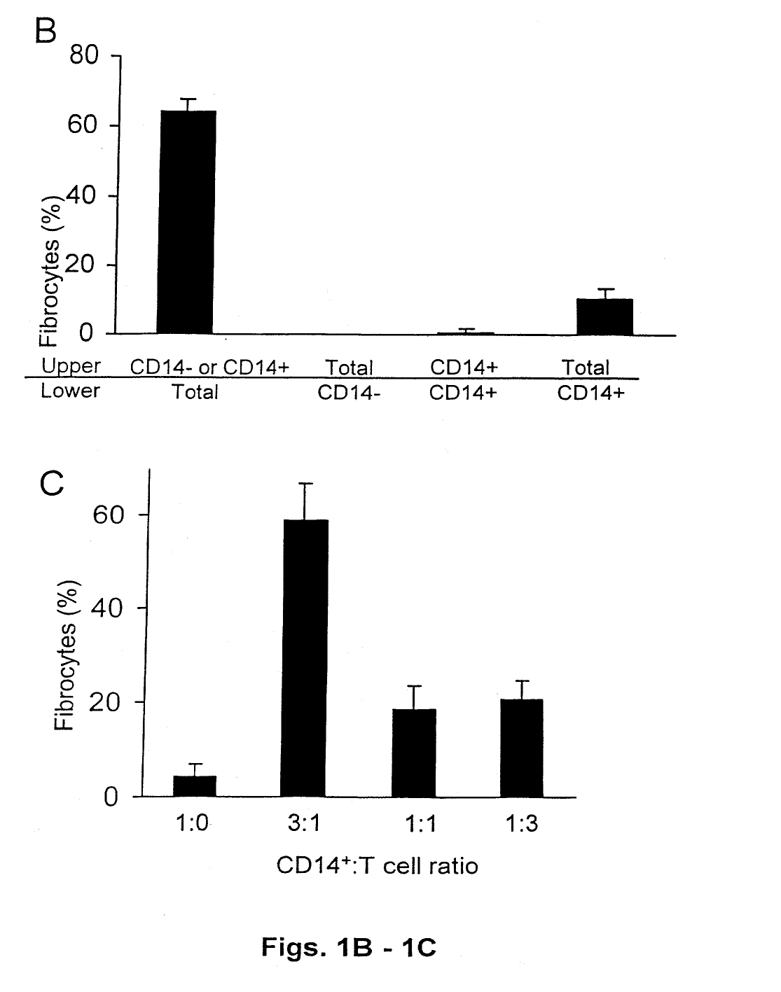

A peripheral blood population consisting predominantly of CD14⁺ cells, but not a CD14⁻ cell population, gives rise to fibrocytes in vitro. To determine the origin of fibrocytes, we analyzed the growth and phenotype of adherent human peripheral blood mononuclear cells cultured on plastic (see FIG. 1A). After standard Ficoll™ separation, the resulting population was approximately 40-50% CD14⁺ cells. Following an overnight adherence step, the adherent cell population (“total”) was >70% CD14⁺ cells exhibiting no detectable collagen I staining, as assessed by flow cytometry (data not shown; 5). We have shown in previous studies that, after 2 weeks, cells in these cultures no longer express CD14, but do express collagen I (5). Importantly, we found that a cell population enriched for “CD14⁺” cells, (i.e. PBMCs depleted of all T or B cells by magnetic beads) gives rise to very few collagen I⁺/CD11b⁺ spindle-shaped fibrocytes after one week of culture (data not shown).

Using Transwell™ culture chambers, we examined the cellular requirements for fibrocyte differentiation (CD11b/Col I⁺) in vitro from circulating blood cell fractions (FIG. 1B). When a “CD14⁻” cell fraction was cultured in the lower well of a Transwell™ plate and total PBMCs were cultured in the top chamber for one week, no fibrocytes appeared in the lower chamber. Similarly, no fibrocytes appeared in the lower chamber when “CD14⁺” cells alone were cultured in the bottom chamber and “CD14⁺” cells or total PBMCs were cultured in the top chamber for one week. By contrast, when “total” PBMCs were cultured in the bottom well of the Transwell™ chamber and either “CD14⁻” cells or “CD14⁺” cells (or media alone-data not shown) were cultured in the top chamber, numerous spindle-shaped fibrocytes (CD11b⁺/Col I⁺) were observed within one week. These data suggest that fibrocyte outgrowth from cultured PBMCs requires cellular interaction between a population of enriched CD14⁺ cells and another peripheral blood cell type or that fibrocyte precursors are only present in the PBMC fraction.

To examine the requirement of cellular interaction, we then added either purified, autologous T or B cells to “CD14⁺” cell cultures in various ratios (CD14⁺:T; 0:1, 1:0, 3:1, 1:1, and 1:3) for 7-10 d and found that co-cultures of “CD14⁺” cells and T cells give rise to fibrocytes (CD11b⁺/Col I⁺) (FIG. 1C). We observed that a CD14⁺ cell:T cell ratio of 3:1 was optimal (FIG. 1C) for culturing fibrocytes. By contrast, no fibrocytes appeared when T cells were cultured alone or in co-cultures of B cells and “CD14⁺” cells or when “CD14⁺” cells were cultured with T cell conditioned media (data not shown). Because fibrocytes do not express T cell markers (CD2, CD3, CD4, CD8) or typical T cell cytokines (IL-2, IL-4, IFNγ), it is unlikely that T cells give rise to fibrocytes.

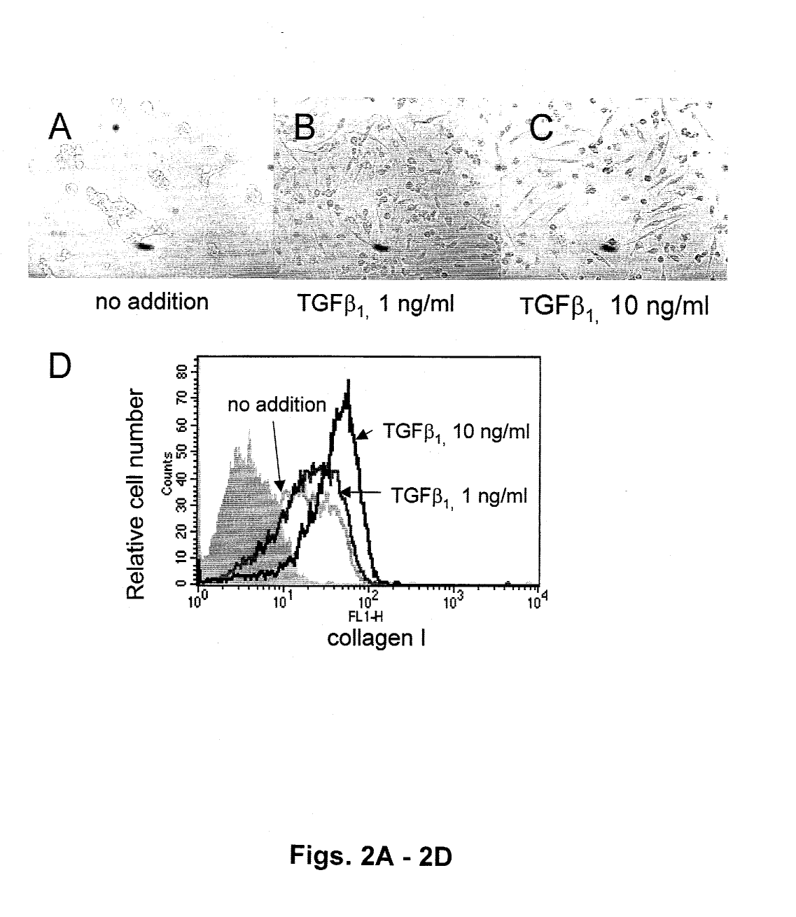

TGFβ₁ accelerates fibrocyte differentiation in vitro. Next, we examined whether TGFβ₁, a cytokine important for fibroblast proliferation and extracellular matrix production could promote the differentiation and accumulation of fibrocytes within PBMC cultures. The addition of TGFβ (1-10 ng/ml) to PBMC cultures on days 3-10 promoted fibrocyte differentiation in vitro, as shown by the enhanced accumulation of cells with spindle-shaped morphology (FIGS. 2A-C). Treatment of these cultures with TGFβ₁ increased the expression of collagen I by fibrocytes within these cultures in a dose-dependent manner (FIG. 2D). The mean fluorescence intensity for collagen I expression was 11, 24, and 63 for fibrocytes in cultures treated with 0, 1, and 10 ng/ml TGFβ₁, respectively (FIG. 2D). These Col I⁺ cells also stained positively for CD11b (data not shown). Furthermore, there was a dose-dependent increase in the number of cells that stained positive for collagen I in response to TGFβ₁ within the cultures, with almost a 40% increase in response to 10 ng/ml TGFβ₁ when compared to untreated cultures. Similar results were observed with fibrocyte preparations from three other donors, each showing 30-45% increase in collagen I expression between 0 and 10 ng/ml TGFβ₁.

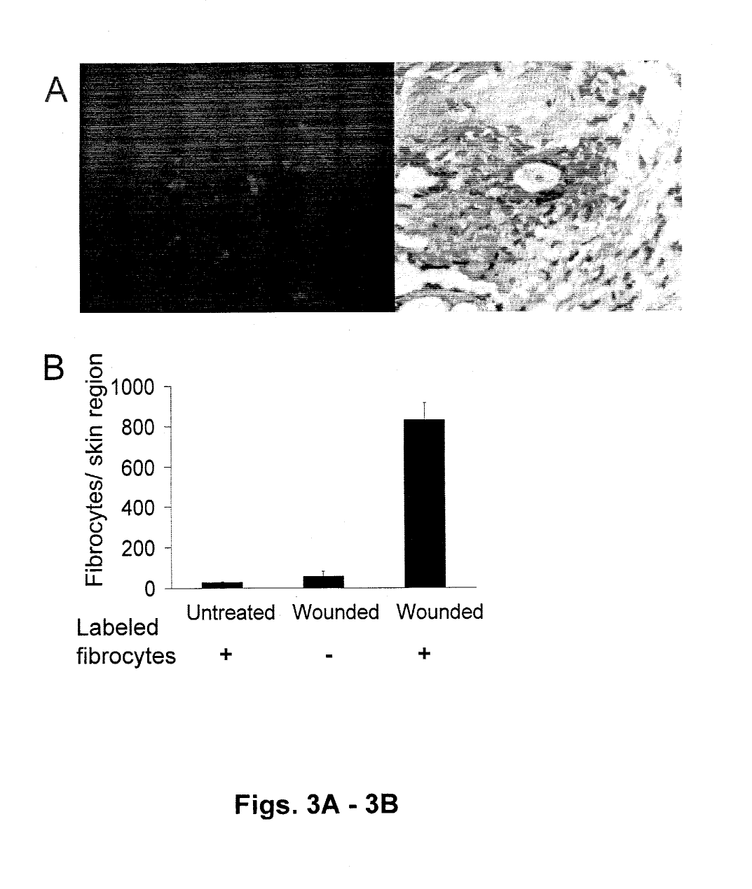

Fibrocytes cultured ex vivo migrate to wound sites. We next sought to quantify the migration into wound sites of transferred cultured, “enriched” fibrocytes using a mouse model system. Cultured, “enriched” mouse fibrocyte preparations (>96% pure) that had been labeled with a fluorescent dye were injected (5×10⁵/mouse) into the tail vein of mice. Immediately, full-thickness skin punch biopsy wounds (5 mm diameter) were made in the dorsal scapular area in some mice. The wound sites (and comparable untreated skin tissue) were excised 4 days later and biopsy specimens were examined for the presence of labeled fibrocytes. As shown in FIG. 3A, numerous fluorescent cells were found by microscopic analysis of the wound tissue at 4 d. Labeled fibrocytes appeared to be located near newly formed blood vessels at the edge of the wound. Using another group of mice (n=3 per group), single cell suspensions were prepared from the excised wound or normal tissue (250 μg/biopsy) and labeled fibrocytes were quantified by flow cytometry. Enumeration of migrated labeled fibrocytes revealed that wounded tissue contained significantly more labeled fibrocytes than a similar area of normal skin taken from the same mouse (FIG. 3B).

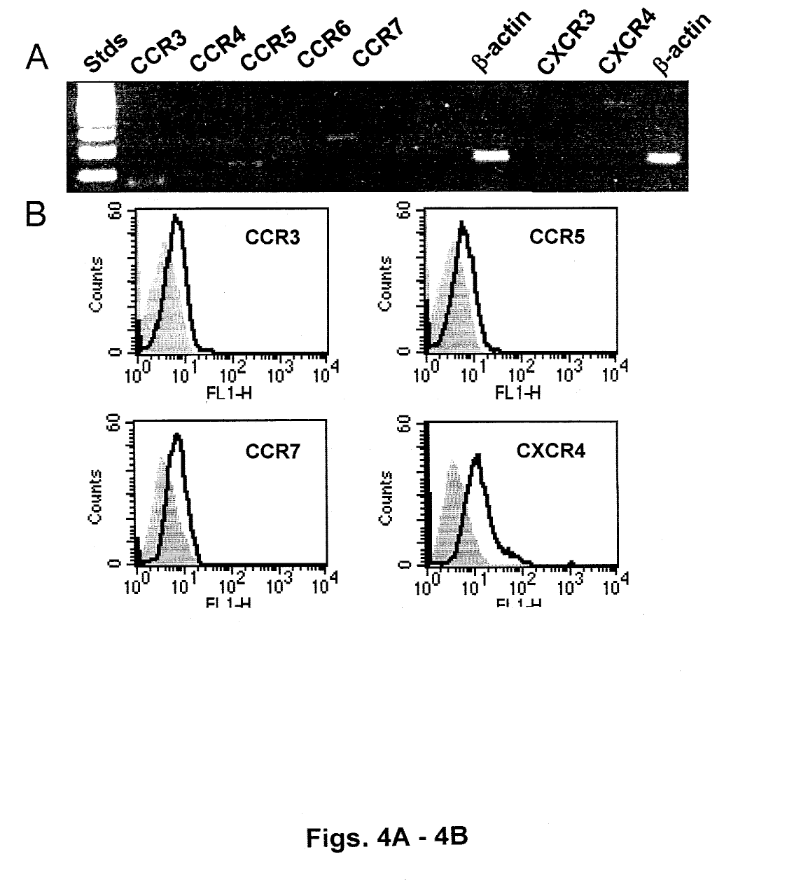

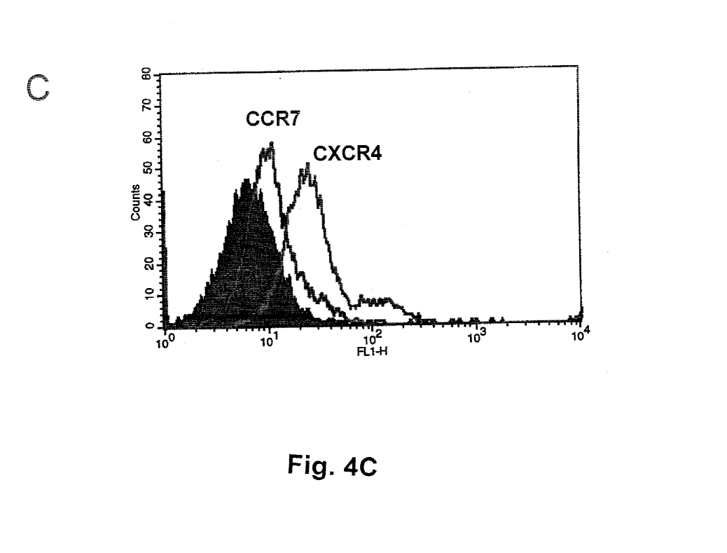

Fibrocytes express functional chemokine receptors and migrate in response to secondary lymphoid chemokine (SLC) in vitro and in vivo. Numerous circulating cells such as, neutrophils, monocytes, and T cells, are known to migrate into cutaneous wound sites. This process is organized, in part, by specific interactions between chemokines and their receptors. We surveyed cultured “enriched” fibrocyte preparations for chemokine receptor mRNA expression by RT-PCR, and found CCR3, −5, −7, and CXCR4 mRNA (FIG. 4A), but not CCR4, CCR6, or CXCR3 mRNA expression. We confirmed CCR3, CCR5, CCR7and CXCR4 protein expression on the surface of human cells “enriched” fibrocyte cultures by flow cytometry (FIG. 4B). Cells from cultured, “enriched” fibrocyte preparations isolated from mouse blood also expressed CCR7 and CXCR4, as analyzed by cytofluorometric analysis (FIG. 4C).

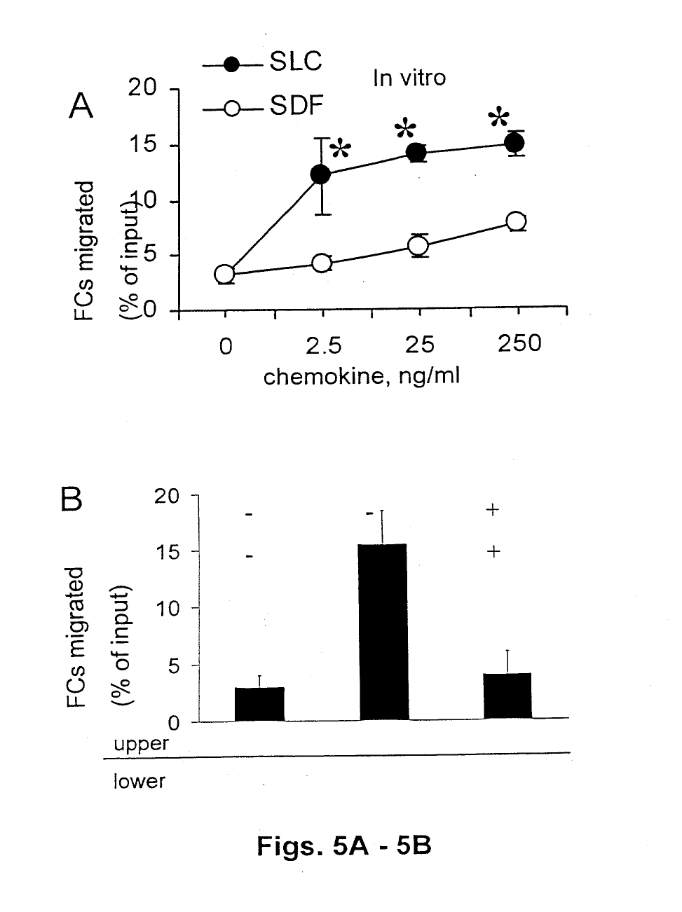

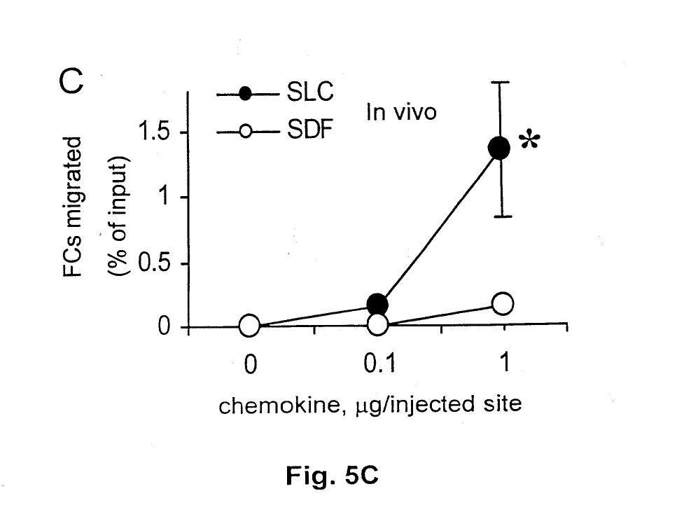

Based on the expression of CCR7, a receptor for SLC, and CXCR4, a receptor for SDF, by populations “enriched” for fibrocytes we used SLC and SDF in an in vitro chemotaxis assay. As shown in FIG. 5A, SLC significantly induced the migration of fibrocytes, whereas SDF did not. Checkerboard analyses confirmed the chemotactic (but not chemokinetic) response to SLC of cell preparations culture and “enriched” fibrocytes (FIG. 5B). Based on these observations, we investigated whether SLC could promote the migration of cells transferred from cultured, “enriched” fibrocyte preparations following an i.d. injection of the chemokine in vivo. Administered at a dose of 1 μg, SLC dramatically induced the accumulation of pre-labeled, ex vivo cultured fibrocytes in the skin area surrounding the i.d. injection site when compared to PBS alone (FIG. 5C). By contrast, SDF injection did not promote fibrocyte chemotaxis in vivo (FIG. 5C). Immunostaining of a 2-day wound site revealed SLC chemokine expression by the vascular endothelium (data not shown). These results suggest that fibrocytes migrate into early wound sites, owing in part to an interaction between vascular endothelium-derived SLC and fibrocyte CCR7.

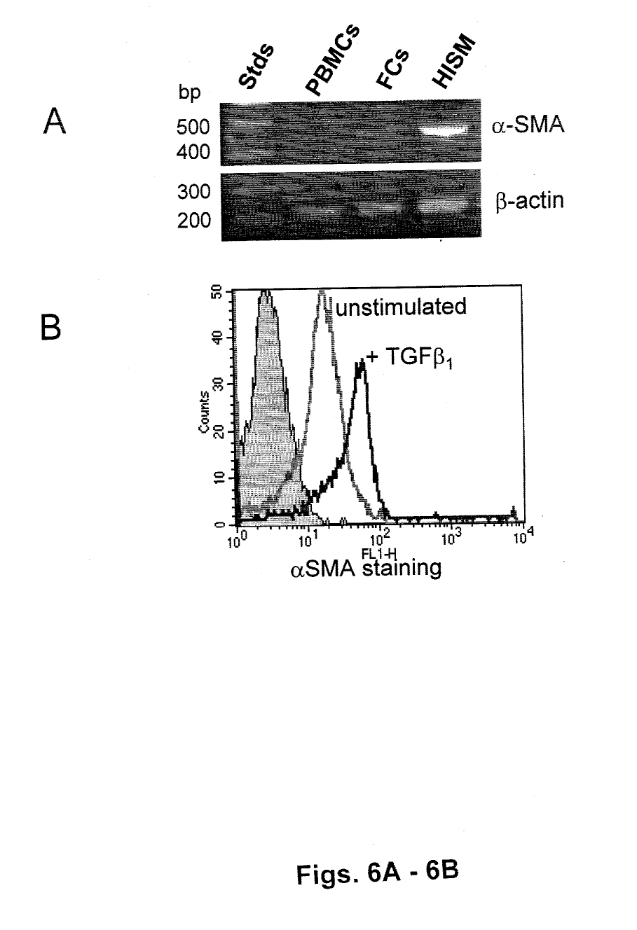

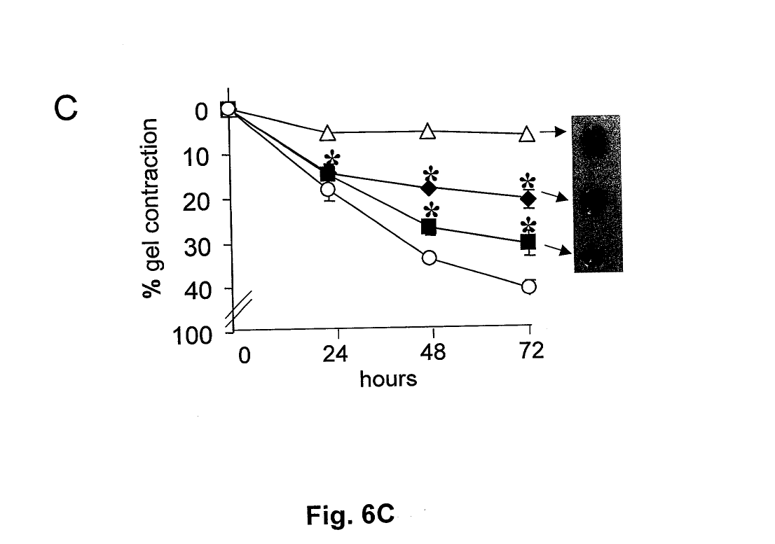

Fibrocytes contract collagen gels. Based on their presence within the wound and their expression of collagen types I and III, we postulated that fibrocytes mediate wound healing and fibrosis. Gabbiani and co-workers have previously described a population of wound-fibroblasts that differentiate into ‘myofibroblasts’ in the presence of TGFβ (17, reviewed in 18). These cells are characterized by expression of αSMA, the activity of contracting collagen gels in vitro, and their proposed role in wound closure, inflammation, and fibrosis (reviewed in 19). Recognizing that TGFβ₁ enhances collagen I expression by cultured fibrocytes (FIG. 2D) and that fibrocytes are present in wound tissue for days (20), we next examined whether cultured, “enriched” fibrocyte preparations express αSMA and exhibit a contractile force. As shown FIG. 6A, unstimulated, cultured, “enriched” fibrocyte preparations were found to express αSMA mRNA, but freshly isolated PBMCs did not. Unstimulated cultured, “enriched” fibrocyte preparations also express αSMA protein, and the addition of TGFβ₁ (10 ng/ml) increased αSMA levels by about four-fold (FIG. 6B). Next, we examined the contractile activity of isolated cultured, “enriched” fibrocyte populations. We found that untreated cultured, “enriched” fibrocyte populations significantly contracted the collagen gels in vitro by ˜20%, whereas PBMCs did not (FIG. 6C). Pretreatment of fibrocytes with TGFβ₁ (10 ng/ml) for 7 days prior to the assay further increased their contractile activity (FIG. 6C). This increase in gel contraction by TGFβ₁-treated fibrocyte cultures correlated with the enhanced expression of αSMA by fibrocytes in response to TGFβ₁.

### METHODS

Mice. BALB/c mice (female, 8-12 wks) were purchased from The Jackson Laboratory (Bar Harbor, Me.). All animal procedures were conducted according to guidelines of the Institutional Animal Care and Use Committee of North Shore University Hospital under an approved protocol.

Antibodies, cytokines, and chemokines. FITC-anti-αSMA mAb was purchased from Sigma (St. Louis, Mo.). Biotinylated rabbit anti-collagen I and biotinylated rabbit IgG were purchased from Rockland Immunochemicals (Gilbersville, Pa.). Anti-mouse CCR3, CCR5, CCR7, or CXCR4 polyclonal antibodies and FITC-anti-goat IgG antibody were purchased from Santa Cruz Biotechnology Inc. (Santa Cruz, Calif.). All other antibodies were purchased from BD PharMingen (San Diego, Calif.). TGFβ₁ (active), secondary lymphoid chemokine (SLC), and stromal-derived cell factor (SDF) were purchased from R&D Systems (Minneapolis, Minn.).

Cells. Fibrocytes (human and mouse) were purified from peripheral blood and cultured as previously described (2,5). Briefly, peripheral blood mononuclear cells (PBMCs) were isolated from human Leukopaks® (purchased from the Long Island Blood Center) by centrifugation over Ficoll/Paque™ (Pharmacia) following the manufacturer's protocol. After two days of culture on tissue culture flasks in DMEM (Life Technologies, Gaithersburg, Md.) supplemented with 20% FBS (HyClone), penicillin, streptomycin, and L-glutamine, non-adherent cells were removed by gentle aspiration and media were replaced. After 10-12 days, adherent cells were lifted by incubation in ice cold 0.05% EDTA (in PBS). The “crude fibrocyte” preparations (approximately 70-80% pure based on collagen I/CD11b staining) then were depleted by immunomagnetic selection of contaminating T cells (˜13%), B cells (˜3%), and monocytes (˜11%) using pan-T, anti-CD2; Pan-B, anti-CD19; and anti-CD14 Dynabeads™, respectively (Dynal, Great Neck, N.Y.). The resultant cultured, “enriched fibrocyte” populations were ≧95% pure based on collagen I/CD11b staining, with T cells and monocytes contributing approximately 3% and 2%, respectively. Typically, between 0.4-5×10⁴ fibrocytes were isolated per ml of human blood.

Mouse peripheral blood mononuclear cells were isolated from BALB/c mouse blood (heparinized) obtained by cardiac puncture following CO₂ asphyxiation. Mouse blood was mixed with PBS (2:1) and layered over Ficoll/Paque™ (Pharmacia) (15 ml blood over 30 ml Ficoll™) and centrifuged according to the manufacturer's protocol. Mouse fibrocytes were cultured from isolated buffy coats in DMEM supplemented with 10% FBS and 10% mouse serum (Sigma), penicillin, streptomycin, and L-glutamine, as previously described (4). After 10-12 days, the adherent “crude” fibrocyte preparations (approximately 75% pure based on collagen I/CD11b staining) were lifted using 0.05% EDTA in PBS and depleted by immunomagnetic selection of contaminating T cells, B cells, and monocytes using pan-T (anti-CD90), pan-B (anti-B220) Dynabeads™ (Dynal), and anti-mouse CD14 attached to Dynabeads™, respectively. Following immunodepletion, the cultured, “enriched” fibrocyte preparations were verified to be ≧95% pure by collagen I⁺/CD11b⁺ staining as determined by flow cytometry. Approximately 0.8-4×10⁴ fibrocytes were purified per ml of mouse blood (˜1-1.2 ml blood per mouse).

Human adult dermal fibroblasts were purchased from Clonetics (San Diego, Calif.) and cultured according to the manufacturer's recommendations. The human intestinal smooth muscle cell line, HISM, was obtained from ATCC (Manassas, Va.) and cultivated according to recommended procedures.

Analysis of fibrocyte differentiation. Initial studies were aimed toward elucidating the cellular origin of peripheral blood-derived fibrocytes. Therefore, we fractionated whole blood supplied as Leukopaks® (shown in FIG. 1A) and cultured the various fractions in vitro. Adherent cells were collected from overnight cultures of human PBMCs (“total”) and CD14⁺ cells were enriched from the PBMC fraction by depletion of T and B cells (“CD14⁺”). “CD14⁻cells”(including all PBMCs except CD14⁺ cells) were purified by depletion of the CD14⁺ cells from the total PBMC preparation. Using the Transwell™ two-chamber system (0.4 μm pore size in separating membrane) (Corning Costar, Cambridge, Mass.), “CD14⁺”, “CD14⁻”, or “total” cells (3×10⁶ cells/ml in DMEM 10% FBS) were cultured in either the upper or lower chambers, as indicated. After 7 days of culture, the cells that grew in the lower well were collected and analyzed for ‘fibrocyte’ differentiation operationally defined by collagen I/CD11b staining in flow cytometry. Similar results were observed with cells prepared from three other donors.

For studies investigating a requirement for T cells in fibrocyte differentiation, the “CD14⁺” cell fraction (see above) was purified from purchased Leukopaks® and cultured with autologous T cells isolated using T cell enrichment columns (R&D). T cell purity was ≧95%, as assessed by flow cytometry using anti-CD3 antibodies (PharMingen). After seven days co-culture, the resulting population was analyzed for the percentage of fibrocytes by collagen I/CD11b staining and flow cytometry. Similar results were observed using fibrocytes isolated from three different donors.

Flow cytometric analysis. For single antibody staining, cells (10⁵ aliquots) were re-suspended in PBS containing 3% BSA and 0.1% sodium azide (FACS buffer) and incubated with the indicated antibodies (or labeled isotype control antibodies) for 30 minutes at 4° C. In cases where the primary antibodies were not labeled, cells were washed and incubated with revealing antibodies diluted in FACS buffer. After washing the cells in FACS buffer, fluorescence data were acquired on a FACSCalibur® flow cytometer (Becton Dickinson, San Jose, Calif.) and analyzed using CELLQuest™ software (Becton Dickinson). At least 5,000 cells were analyzed per condition. To analyze preparations for collagen I/CD11b staining, cells were prepared as above and first incubated in FACS buffer containing biotinylated collagen I antibody (or biotinylated rabbit control IgG), then washed and incubated sequentially in FACS buffer containing FITC-strepavidin (PharMingen) and PE-CD11b (PharMingen). Intracellular staining for αSMA was performed as previously described (6,7). Briefly, cells were fixed and permeabilized using the Cytoperm/Cytofix™ kit (PharMingen) according to the manufacturer's recommendations and incubated with FITC-anti-αSMA mAb (Sigma).

Fibrocyte migration in vivo using a wound model. Cultured, “enriched” peripheral blood-derived mouse fibrocytes (>96% pure) were stained with a membrane-inserting red dye, PKH-26 (Sigma), following the manufacturer's protocol. Labeling efficiency, assessed by flow cytometry, and viability, assessed by trypan blue exclusion were >85%. PKH-labeled cell preparations (5×10⁵) in 100 μl PBS were administered into the tail vein (i.v.) of BALB/c mice (n=2 per group per group). Immediately following injection of the labeled “enriched” fibrocyte preparations, a full-thickness round skin wound (5 mm diameter) was made in the dorsal subscapular area of each recipient mouse by excision with skin punch equipment, as previously described (8). Wound sites were removed four days later and examined for the presence of fluorescent fibrocyte cells by microscopic analysis of thin frozen sections and by quantitative flow cytometric analysis following proteolytic digestion of biopsied material. For quantitative flow cytometric analysis, excised skin (250 μg biopsy per animal) was chopped into small fragments, then incubated for 1 h at 37° C. in RPMI containing 10% FBS, 2 mg/ml collagenase and 20 μg/ml DNase I. The resulting single cell suspension was examined by flow cytometry to determine the number of fluorescent fibrocytes present using calibration beads as previously described (15).

RT-PCR. Total RNA was isolated from cultured, “enriched” fibrocyte preparations (>95% purity) using RNAzol B (Tel-Test, Friendswood, Tex.). The cDNA was prepared from 1.0 μg of RNA using 0.25 μg of oligo-(dT),₁₂₋₁₈ and MMLV reverse transcriptase following the protocol supplied by the manufacturer (Gibco). Two μl aliquots of cDNA were amplified by PCR using Supermix™ (Gibco) in a Perkin Elmer model 9600 thermal cycler using specific primers PCR pairs, as previously described: αSMA (9); CCR3 (10); CCR4, CCR5, and CXCR3 (11); CCR6 (12); CCR7 (13); CXCR4 (14); β-actin the sense primer was 5′-GTGGGGCGCCCCAGGCACCA-3′, and the antisense primer was 5′-CTCCTTAATGTCACGCACGATTTC-3′. Thermal cycling (25-30 cycles; in 25 μl) was performed as follows: denaturation at 94° C. for 0.5 min; annealing at 55° C. for 0.5 min; and extension at 72° C. for 1 min. PCR products were separated by electrophoresis through 2% agarose gels and viewed under UV light after ethidium bromide staining. To control for potential genomic DNA contamination, PCR reactions were performed without the RT step and no DNA amplification products were detected.

In vitro fibrocyte chemotaxis assay. Chemotaxis assays were performed using Costar Transwell™ inserts (8 μm pore size) according to the manufacturer's protocol. Cultured, “enriched fibrocytes” (>95% pure) were resuspended at 1×10⁶ cells/ml in DMEM containing 0.1% BSA. Media alone (negative control) or media containing SLC or SDF (600 μl to provide a final chemokine concentration of 2.5-250 ng/ml as indicated) was added to individual wells of a 24-well plate. Transwell™ devices then were inserted, and the fibrocytes (100 μl) were layered on top of the membrane (n=3 wells per condition). After 3 hrs, the transmigrated cells were collected and counted by flow cytometry using calibration beads (Coulter, Miami, Fla.), as previously described (15). Similar results were observed with 2 additional donors. For checkerboard analysis of SLC-directed chemotaxis of fibrocytes, 100 ng/ml SLC was added to either the top or bottom chamber alone, and to both the bottom and top chambers, as indicated in FIG. 5B.

In vivo fibrocytes chemotaxis assay. Immediately following tail vein injection of PKH-labeled “enriched fibrocyte preparations” (>94% pure; 5×10⁵ cells/mouse), BALC/c mice received either an i.d. injection of SLC, SDF (0.1 or 1 μg in 50 μl) or PBS alone in the scapular region of the back (shaved). The injected site was excised 4 hrs later and proteolytically digested to produce a single cell suspension (as described above). The number of labeled fibrocytes per biopsy sample (250 μg) was estimated by flow cytometry using calibration beads (15). This experiment was repeated twice with similar results.

Collagen lattice contraction assay. Cellular collagen gel contraction assays were performed as previously described (16). Overnight adherent PBMC cultures, 10 day old “enriched fibrocyte preparations” (≧95% pure) previously cultured in the absence or presence of TGFβ₁ (10 ng/ml for 7 days prior to experiment), or normal human dermal fibroblasts were lifted using cold EDTA/PBS solution. A collagen solution in DMEM was prepared from rat tail collagen type I according to the manufacturer's instructions, and combined with cells at 2×10⁵/ml (n=3 per cell type). The collagen/cell mixture (400 μl/well) was dispensed into culture plates and allowed to polymerize at 37 C. for 30 min. Immediately after polymerization, 2 ml of DMEM containing 10% FBS were added to each well. The gels then were detached from the wells by gently shaking the culture plates at various time points (0, 24, 48 and 72 h) and the longest and the shortest diameters of each gel were measured. The mean of the linear measurements (n=3 for each sample) taken at each time point was used to estimate the contractility of the cells. The data are presented as % gel contraction. This experiment was repeated twice with similar results using cells obtained from different donors.

As will be apparent to a skilled worker in the field of the invention, numerous modifications and variations of the present invention are possible in light of the above teachings. It is therefore to be understood that the invention may be practiced otherwise than as specifically described herein.

### REFERENCE LIST

1. Dunphy et al., “The Fibroblast—A Ubiquitous Ally for the Surgeon”, NEJM, 268, 1367 (1963).

2. Bucala, et al., “Circulating Fibrocytes Define a New Leukocyte Subpopulation that Mediates Tissue Repair”, Mol. Med., 1, 71 (1994).

3. Chesney, et al.,“The Peripheral Blood Fibrocyte is a Potent Antigen-Presenting Cell Capable of Priming Naive T cells in situ”, Proc. Natl. Acad. Sci. U.S.A, 94, 6307 (1997).

4. Grab, et al., “Interaction of Borrelia Burgdorferi with Peripheral Blood Fibrocytes,Antigen-Presenting Cells with the Potential for Connective Tissue Targeting”, Mol. Med., 5, 46 (1999).

5. Chesney et al., “Regulated Production of Type I Collagen and Inflammatory Cytokines by Peripheral Blood Fibrocytes”, J. Immunol., 160:419 (1998).

6. Arora et al., “The Dletion of Tansforming Gowth Factor-Beta-Induced Myofibroblasts Depends on Growth Conditions and Actin organization”, Am. J. Pathol., 155, 2087 (1999).

7. Arora, et al., “The Compliance of Collagen Gels Regulates Transforming Growth Ffactor-Beta Induction of Alpha-Smooth Muscle actin in Fibroblasts”, Am. J. Pathol., 154, 871 (1999).

8. Matsuda, et al., “Role of Nerve Growth Factor in Cutaneous Wound Healing: Accelerating Effects in Normal and Healing-Impaired Diabetic Mice”, J. Exp. Med., 187, 297 (1998).

9. Adachi et al.,“Skeletal and Smooth Muscle Alpha-Actin mRNA in Endomyocardial Biopsy Samples of Dilated Cardiomyopathy Patients”, Life Sci., 63,1779 (1998).

10. Vestergaard, et al., “Overproduction of Th2-specific Chemokines in NC/Nga Mice Exhibiting Atopic Dermatitis-Like Lesions, J. Clin. Invest., 104, 1097 (1999).

11. Yoneyama et al., “Pivotal Role of TARC, a CC Chemokine, in Bacteria-Induced Fulminant Hepatic Failure in Mice”, J. Clin. Invest., 102, 1933 (1998).

12. Varona, et al., “Molecular Cloning, Functional Characterization and mRNA Expression Analysis of the Murine Chemokine Receptor CCR6 and its Specific Ligand MIP-3alpha”, FEBS Lett., 440,188 (1998).

13. Saeki et al., “Cutting Edge: Secondary Lymphoid-Tissue Chemokine (SLC) and CC Chemokine Receptor 7 (CCR7 ) Participate in the Emigration Pathway of Mature Dendritic Cells from the Skin to Regional Lymph Nodes”, J. Immunol., 162, 2472 (1999).

14. Luos et al., “Chemokine Amplification in Mesangial Cells”, J. Immunol., 163, 3985 (1999).

15. Bleul, et al., “A Highly EfficaciousLlymphocyte Chemoattractant,H Stromal Cell-Derived Factor 1 (SDF-1)”, J. Exp. Med., 184, 1101 (1996).

16. Racine-Samson, et al., “The role of Alpha1beta1 Integrin in Wound Contraction. A Quantitative Aanalysis of Liver Myofibroblasts in vivo and in Primary Culture”, J. Biol. Chem., 272, 30911 (1997).

17. Vaughan. et al., “Transforming Growth Factor-Beta1 Promotes the Morphological and Functional Differentiation of the Myofibroblas”, Exp. Cell Res., 257, 180 (2000).

18. Serini et al., “Mechanisms of Myofibroblast Activity and Phenotypic Modulation”, Exp. Cell Res., 250, 273. (1999).

19. Powell, et al., “Myofibroblasts. I. Paracrine Cells Important in Health and Disease”, Am. J. Physiol, 277, C1 (1999).

20. Chesney, et al., “Regulated Production of Type I Collagen and Inflammatory Cytokines by Peripheral Blood Fibrocytes”, J. Immunol., 160, 419 (1998).

21. Chesney et al., “Peripheral Blood Fibrocytes: Novel Fibroblast-Like Cells that Present Antigen and Mediate Tissue Repair:, Biochem. Soc. Trans., 25, 520 (1997).

22. Xu et al., “Dendritic Cells Differentiated from Human Monocytes Through a Combination of IL-4, GM-CSF and IFN-Gamma Exhibit Phenotype and Function of Blood Dendritic Cells:, Adv. Exp. Med. Biol., 378, 75 (1995).

23. Pickl et al., “Molecular and Functional Characteristics of Dendritic Cells Generated from Highly Purified CD14⁺ Peripheral Blood Monocytes”, J. Immunol., 157, 3850 (1996).

24. Zhou, et al., “CD14⁺ Blood Monocytes can Differentiate into Functionally Mature CD83⁺ Dendritic Cells”, Proc. Natl. Acad. Sci.U.S.A., 93, 2588 (1996).

25. Chapuis, et al., “Differentiation of Human Dendritic Cells from Monocytes in vitro”, Eur. J. Immunol., 27, 431 (1997).

26. Shreedhar, et al., “Dendritic Cells Require T Cells for Functional Maturation in vivo”, Immunity, 11, 625 (1999).

27. Kalinski et al., “Final Maturation of Dendritic Cells is Associated with Impaired Responsiveness to IFN-Gamma and to Bacterial IL-12 Inducers: Decreased Ability of Mature Dendritic Cells to Produce IL-12 During the Interaction with Th Cells”, J. Immunol., 162, 3231 (1999).

28. Branton, et al., “TGF-Beta and Fibrosis”, Microbes. Infect., 1, 1349 (1999).

29. Chu, et al., “Up-Regulation by Human Recombinant Transforming Growth Factor Beta-1 of Collagen Production in Cultured Dermal Fibroblasts is Mediated by the Inhibition of Nitric Oxide Signaling”, J. Am. Coll. Surg., 188, 271 (1999).

30. Zhang, et al., “Inhibition of Myofibroblast Apoptosis by Transforming Growth Factor Beta(1)”, Am. J. Respir. Cell Mol. Biol., 21, 658 (1999).

31. Wang et al., “Exogenous Transforming Growth Factor Beta(2) Modulates Collagen I and Collagen III Synthesis in Proliferative Scar Xenografts in Nude rats”, J. Surg. Res., 87, 194 (1999).

32. Cromack, et al., “Transforming Growth Factor Beta Levels in Rat Wound Chambers”, J. Surg. Res., 42, 622 (1997).

33. Benn, et al., “Particle-Mediated Gene Transfer with Transforming Growth Factor-Beta1 cDNAs Enhances Wound Repair in Rat Skin”, J. Clin. Invest., 98, 2894 (1996).

34. Jelaska, et al., “Fibroblast Heterogeneity in Physiological Conditions and Fibrotic Disease”, Springer Semin. Immunopathol., 21, 385 (1999).

35. McCormick, et al., “Anti-TGF-Beta Treatment Prevents Skin and Lung Fibrosis in Murine Sclerodermatous Graft-Versus-Host Disease: A Model for Human Scleroderma”, J. Immunol., 163, 693 (1999).

36. Sime, et al., “Adenovector-Mediated Gene Transfer of Active Transforming Growth Factor-Beta1 Induces Prolonged Severe Fibrosis in Rat Lung”, J. Clin. Invest., 100, 768 (1997).

37. Gauldie et al., “Transforming Growth Factor-Beta Gene Transfer to the Lung Induces Myofibroblast Presence and Pulmonary Fibrosis”, Curr. Top. Pathol., 93, 35 (1999).

38. Saeki, et al., “Cutting Edge: Secondary Lymphoid-Tissue Chemokine (SLC) and CC Chemokine Receptor 7 (CCR7) Participate in the Emigration Pathway of Mature Dendritic Cells from the Skin to Regional Lymph Nodes”, J. Immunol., 162, 2472 (1999).

39. Hjelmstrom. et al., “Lymphoid Tissue Homing Chemokines are Expressed in Chronic Inflammation”, Am. J. Pathol., 156, 1133 (2000).

40. Gunn, et al., “Mice Lacking Expression of Secondary Lymphoid Organ Chemokine have Defects in Lymphocyte Homing and Dendritic Cell Localization”, J. Exp. Med., 189, 451 (1999).

41. Nakano, et al., “A Novel Mutant Gene Involved in T-Lymphocyte-Specific Homing into Peripheral Lymphoid Organs on Mouse Chromosome 4”, Blood, 91, 2886 (1998).

What is claimed is:

1. A method for producing fibrocytes comprising contacting a population of human peripheral blood mononuclear cells (PBMC) comprising at least about 40% CD14⁺ cells with autologous T cells, thereby inducing differentiation of fibrocytes from precursors in the PBMC population.

2. The method of claim 1, wherein said contacting is for a period of about 7 to about 10 days.

3. The method of claim 1, wherein said population comprising at least about 40% CD14⁺ cells is provided by cultivation of an adherent PBMC population on a solid substrate.

4. The method of claim 3, wherein said population comprises at least about 70% CD14⁺ cells.

5. The method of claim 1, wherein said population comprising CD14⁺ cells is purified by removal of T or B cell populations using antibodies to cell surface antigens.

6. A method for producing fibrocytes by inducing differentiation of fibrocytes from a PBMC population, comprising contacting said PBMC population with a form of TGFβ.

7. The method of claim 6, wherein said form of TGFβ is TGFβ₁.

8. The method of claim 7, wherein said PBMC population is cultured with 1-10 ng/ml TGFβ₁ for at least about 3 days.

9. The method of claim 6, wherein said PBMC population is in contact with T cells during said contacting with a form of TGFβ.

10. A method of treating a wound in a mammalian subject comprising administering fibrocytes in combination with a form of TGFβ.

11. The method of claim 10, wherein said form of TGFβ is TGFβ₁.

12. The method of claim 10, wherein said fibrocytes and TGFβ₁ are administered in a single composition.

13. The method of claim 10, wherein said fibrocytes and TGFβ₁ are administered in separate compositions.

14. The method of claim 10, wherein said fibrocytes are administered systemically or locally

15. A method for purifying or enriching for fibrocytes comprising exposing a fibrocyte-containing mixed cell population to a gradient of an agonist of the CCR7 chemokine receptor such that fibrocytes separate themselves from other cell types in the mixed cell population by chemotactic response toward said agonist.

16. The method of claim 15, wherein said agonist of the CCR7 chemokine receptor is secondary lymphoid chemokine (SLC).

17. A method for attracting or targeting fibrocytes to a wound in a mammalian subject comprising administering an agonist of the CCR7 chemokine receptor to the subject at or near the site of the wound.

18. The method of claim 17, wherein said agonist of the CCR7 chemokine receptor is SLC.

19. The method of claim 17, wherein said agonist of the CCR7 chemokine receptor is administered locally, intradermally, subdermally or intraperitoneally at or near the site of an unexposed wound.

20. The method of claim 18, wherein said SLC is administered in a unit dosage of from about 100 ng to about 1 mg/dose at least once a day for at least about three days or until the desired healing is obtained.

21. The method of claim 17, further comprising administering fibrocytes to said subject having a wound before, after or concurrently with administering an agonist of the CCR7 chemokine receptor to said subject.

22. A method of decreasing undesired effects of fibrocytes comprising administering an inhibitor of fibrocyte activity a mammalian subject, wherein said inhibitor is selected from the group consisting of agents that interfere with stimulation of fibrocyte differentiation by T cells, agents that interfere with stimulation of fibrocyte differentiation by TGFβ₁, and agents that interfere with attraction of fibrocytes by SLC, or a combination of agents selected from said group.

23. The method of claim 22, wherein said undesired effects of fibrocytes comprise undesired wound fibrosis and said subject has a wound that exhibits or is expected to exhibit undesired fibrosis.
